(19) United States
(12) Patent Application Publication
(10) Pub. No.: US 2017/0245777 A1
(43) Pub. Date: Aug. 31, 2017

(54) SYSTEM AND METHOD FOR DETERMINING NOCICEPTIVE WITHDRAWAL REFLEX THRESHOLD AND QUANTIFYING A REFLEX RECEPTIVE FIELD

(71) Applicant: AALBORG UNIVERSITET, Aalborg Øst (DK)

(72) Inventors: Ole KÆSELER ANDERSEN, Skørping (DK); Michael BRUN JENSEN, Valby (DK); José Alberto BIURRUN MANRESA, Aalborg Øst (DK)

(73) Assignee: AALBORG UNIVERSITET, Aalborg Øst (DK)

(21) Appl. No.: 15/516,366

(22) PCT Filed: Oct. 2, 2015
(86) PCT No.: PCT/EP2015/072867

§ 371 Date: Mar. 31, 2017

(30) Foreign Application Priority Data
Oct. 3, 2014 (DK) PA 2014 00565

(51) Int. Cl. A61B 5/0492; A61B 5/04; A61B 5/00

(52) U.S. Cl. CPC A61B 5/0492; A61B 5/4047; A61B 5/4827; A61B 5/04004

(57) ABSTRACT

According to an aspect of the present inventive concept there is provided a method for determining a nociceptive withdrawal reflex threshold of a person, the method comprising: applying a plurality of stimulations to a person with different stimulation intensity levels in accordance with a stimulation intensity algorithm, wherein the plurality of stimulations comprises at least one stimulation having an intensity level provoking a spinal cord withdrawal reflex in the person, recording electromyographic data representative of muscle activity of the person in response to each stimulation of the plurality of stimulations, and processing the stimulation intensity levels and the electromyographic data according to a threshold calculation algorithm to calculate a measure of the nociceptive withdrawal reflex threshold of the person. There is also provided a corresponding system.

### TECHNICAL FIELD

The present inventive concept generally relates to determining a nociceptive withdrawal reflex threshold of a person, quantifying a reflex receptive field and classifying a person with respect to pain hypersensitivity. In particular the inventive concept relates to methods, a system and a computer program product therefor.

### BACKGROUND

The nociceptive withdrawal reflex (NWR) is an autonomic reaction responsible for moving the limbs away from potential harmful stimuli. Several studies have shown that NWR thresholds (NWR-T) are not influenced by emotional state e.g. anxiety under standard testing conditions, unlike subjective pain reports [12,26]. The NWR may be modulated by supra spinal centers during for example attention or distraction [6,18] and also by temporary or permanent plastic changes in neuronal excitability in the afferent pathways following persistent nociceptive input [17]. Indeed, a recent study showed that injection of capsaicin (a pain model mimicking central sensitization processes) induced significant changes of the NWR both in complete spinal cord injured patients and in healthy control subjects [3]. The NWR is thus considered a valid surrogate measure of the excitability of the nociceptive system.

The NWR responses depend on the stimulation site [1]; so instead of assessing NWR-T to stimulation at one single site, several studies have evaluated the reflex receptive field (RRF), which is defined as the skin area from which a reflex response involving a specific muscle can be elicited by painful stimulation [3,5,21,23,24]. Such a topographical approach contains additional information that can be extracted using spatial analyses, e.g. quantification of RRF size in order to assess levels of spinal excitability. Two alternative methods for quantification of RRF areas have been published, which evaluate the reflex response to painful electrical stimulation randomly distributed at a number of sites under the sole of the foot [21,23]. Both of these methods are objective in the sense that the outcome measure (NWR size or probability of NWR occurrence, respectively) in response to a given painful stimuli does not directly rely on conscious decisions from neither the subject nor the investigator. However, the two methods for RRF area quantification do include an element of subjectivity. The intensities of the painful stimulations eliciting the NWR are defined in relation to a subjectively identified pain threshold for each of the stimulation sites. Such subjective pain reports may be influenced by the emotional state of the subject [12,26]. Furthermore, the precision of existing methods for RRF quantification is limited by the large variability present in the measurements.

### SUMMARY OF THE INVENTIVE CONCEPT

In view of the above, a general objective of the present inventive concept is to provide a system and a method enabling an objective determination of a nociceptive withdrawal reflex threshold (NWR-T) of a person. A further object is to provide a system and a method enabling objective quantification of a reflex receptive field (RRF). Further objects may be understood from the following.

These objects of the invention are at least partly met by the invention as defined in the independent claims. Preferred embodiments are set out in the dependent claims.

According to an aspect of the present inventive concept there is provided a system for determining an NWR-T of a person, the system comprising:
a stimulator arranged to generate a stimulation in response to a control signal, wherein an intensity level of the stimulation is adjustable in accordance with information in the control signal,
an electromyographic sensor arranged to sense an electromyographic signal and output electromyographic data representative of muscle activity of the person, and
a processor system arranged for connection to the stimulator and the electromyographic sensor,
wherein the processor system is arranged to provide a set of control signals to the stimulator, the set of control signals being adapted to control the stimulator to provide a set of stimulations with different stimulation intensity levels to the person, wherein at least one stimulation of said set of stimulations has an intensity level arranged to provoke a spinal cord withdrawal reflex in the person,
wherein the processor system is arranged to receive electromyographic data representative of muscle activity of the person in response to each stimulation of said set of stimulations, and
wherein the processor system is arranged to process the stimulation intensity levels and said electromyographic data representative of muscle activity of the person in response to each stimulation to determine a measure of the NWR-T of the person.

By calculating a measure of the person's NWR-T in accordance with the inventive system a measure of the person's NWR-T may be estimated without relying on subjective pain reports from the person. Furthermore, the NWR-T may be determined without any subjective assessment or manual involvement by an investigator or tester. The inventive system may hence advantageously be easily and reliably applied by e.g. nursing staff at e.g. a hospital, a pain clinic, in clinical research or the like.

Objective assessment of a person's NWR-T may find use both in diagnosis of pain conditions, as well as in assessment of treatment efficacy (such as pain killers). Furthermore, an objectively established NWR-T of a person enables an objective assessment of an RRF of the person, as will be further described in the below.

According to one embodiment the processor system is arranged to determine measures of an NWR-T for a plurality of different stimulation positions on the person, such as a plurality of positions on the person's foot sole. Thereby, NWR-T measures of the person may be estimated for a plurality of different positions on a skin area of the person.

According to one embodiment the processor system is programmed to select the plurality of different stimulation positions in a randomized order. This may improve the accuracy of the estimate(s) since less involvement of a tester is needed and also since there may be a lower risk for the person's expectations to influence the muscle activity.

According to one embodiment the processor system is arranged to generate an output indicative of an RRF of the person using the measures of nociceptive reflex thresholds for the plurality of different stimulation positions on the person. By the measures of NWR-T of the person being estimated in an objective manner, in accordance with the above, also the RRF of the person may be objectively estimated. The output may be referred to as an RRF threshold map.

According to one embodiment the processor system is arranged to generate the output indicative of the RRF of the person using a two-dimensional interpolation of the determined measures of NWR-T for the plurality of stimulation positions. Thereby, the RRF of the person may be identified for a continuous skin portion of the person based on only a discrete number of stimulation positions. The output may be referred to as an interpolated RRF threshold map.

According to one embodiment the processor system is arranged to compute a quantity of the RRF indicative of a surface area of a skin portion of the person including the stimulation positions, which surface area presents an NWR-T exceeding a threshold. Alternatively, the processor system is arranged to compute a quantity of the RRF indicative of a fraction of a surface area of the person including the stimulation positions, which fraction presents an NWR-T exceeding a threshold. The threshold may be a relative or an absolute threshold. The threshold may be based on the measures of NWR-T for the plurality of different stimulation positions. The threshold may also be a predetermined threshold. The surface area may for example be a sole of the foot of the person.

The processor system may further be arranged to generate an output indicative of a quantity of an RRF of the person using the measures of nociceptive reflex thresholds for the plurality of different stimulation positions on the person.

The processor system may be arranged to compute a quantity of an RRF of the person using a two-dimensional interpolation of the determined measures of NWR-T for the plurality of stimulation positions.

According to one embodiment the stimulator comprises a plurality of individually controllable stimulator electrodes arranged to be spatially distributed, in relation to each other, to provide stimulation on different positions on the person's skin, such as the person's foot sole. This configuration simplifies measurements for a plurality of positions.

According to one embodiment the individually controllable stimulator electrodes are controllable by the processor system in accordance with a spatial selection algorithm. Thereby, the processor system may control via which electrode a stimulation is to be applied to the person.

According to one embodiment the electromyographic sensor comprises a plurality of surface electromyographic electrodes. Plural electromyographic sensors enables an accurate and non-invasive sensing of muscle activity.

According to one embodiment the electromyographic sensor comprises at least 3 surface electromyographic electrodes, such as the system being arranged to apply a spatial filtering technique to output signals of a plurality of spatially different positioned surface electromyographic electrodes, wherein the system is arranged to apply a cross talk reduction technique to output signals of a plurality of spatially different positioned surface electromyographic electrodes to reduce cross talk between concurrently active muscles. Using 3 surface electromyographic electrodes (in combination with a reference electrode) enables sensing of both a single- and two double-differential electromyographic signals. Acquisition of these three signals improves the ability to identify signals due to muscle activity. In other words the electromyographic sensor may include at least four surface electrodes, wherein three of said surface electrodes are configured as measurement electrodes of electromyographic activity (e.g. in a tripolar configuration) and one of said surface electrodes is configured as a reference electrode.

According to one embodiment the processor system is arranged to, for at least a subset of said set of stimulations, calculate a respective interval peak z-score using the electromyographic data representative of muscle activity of the person in response to the respective stimulation. An interval peak z-score enables reliable detection of muscle activity in response to a stimulation.

According to one embodiment the processor system is arranged to, for at least a subset of said set of stimulations, perform a respective conduction velocity analysis of the electromyographic data representative of muscle activity of the person in response to the respective stimulation. Conduction velocity analysis makes it possible to discern between elevated electromyographic signals due to muscle activity on the one hand and due to cross talk on the other hand.

According to one embodiment the processor system is arranged to determine said set of control signals using an up-down staircase method, such as an interleaved up-down staircase method. This enables accurate probing of the NWR-T of the person while limiting the number of painful stimulations applied to the person.

According to another aspect of the present inventive concept there is provided a method for determining an NWR-T of a person, the method comprising:
applying a plurality of stimulations to a person with different stimulation intensity levels in accordance with a stimulation intensity algorithm, wherein the plurality of stimulations comprises at least one stimulation having an intensity level provoking a spinal cord withdrawal reflex in the person,
recording electromyographic data representative of muscle activity of the person in response to each stimulation of the plurality of stimulations, and
processing the stimulation intensity levels and the electromyographic data according to a threshold calculation algorithm to calculate a measure of the NWR-T of the person.

The details and advantages discussed in connection with the system-aspect and the embodiments thereof apply correspondingly to the method wherein reference is made to the above discussion.

According to yet another aspect, there is provided a method for quantifying an RRF of a person, the method comprising calculating a plurality of measures of NWR-T according to the method of the above aspect for a plurality of different stimulation positions of the RRF of the person, and calculating a quantity measure of the RRF of a person according to a predetermined algorithm.

The details and advantages discussed in connection with the system-aspect and the embodiments thereof apply correspondingly to the method wherein reference is made to the above discussion.

According to yet another aspect, there is provided a method for classifying a person with respect to pain hypersensitivity, the method comprising applying the method of any of the above-mentioned aspects on the person, and comparing a result thereof with a reference value.

According to yet another aspect, there is provided computer program product comprising instructions arranged to perform the method according to any of the above-mentioned aspects and embodiments when executed on a processor system.

According to a further aspect of the present inventive concept there is provided a system for determining a nociceptive withdrawal reflex threshold of a person, such as a system for quantifying a reflex receptive field of a person based on determined nociceptive withdrawal reflex thresholds, the system comprising:
a stimulator arranged to generate a stimulation in response to a control signal, wherein an intensity level of the stimulation is adjustable in accordance with information in the control signal, and wherein the stimulator is arranged to provide a stimulation with an intensity level to provoke a spinal cord withdrawal reflex in the person,
an electromyographic sensor arranged to sense and output at least one electromyographic related parameter representative of muscle activity of the person in response to the stimulation, and
a processor system arranged for connection to the stimulator and the electromyographic sensor, wherein the processor system comprises a processor programmed to execute a stimulation intensity algorithm and a reflex detection algorithm,
wherein the processor system is arranged to generate a set of control signals to the stimulator in accordance with the stimulation intensity algorithm, so as to provide a set of stimulations with different stimulation intensity levels to the person, wherein at least one stimulation has an intensity level arranged to provoke a spinal cord withdrawal reflex in the person,
wherein the processor system is arranged to receive a set of electromyographic related parameter(s) representative of muscle activity in response to said set of stimulations, and
wherein the processor system is arranged to apply the reflex detection algorithm to calculate a measure of the person's nociceptive withdrawal reflex threshold in response to stimulation intensity levels of the set of stimulations and the corresponding set of electromyographic related parameter(s) representative of muscle activity.

According to one embodiment the processor system is arranged to determine measures of nociceptive withdrawal reflex thresholds for a plurality of different stimulation positions on the person, such as a plurality of positions on the person's foot sole. The processor system may be programmed to select the plurality of different stimulation positions in a randomized order.

According to one embodiment the processor system is arranged to generate an output indicative of a quantity of a reflex receptive field of the person in response to the measures of nociceptive reflex thresholds for the plurality of different stimulation positions on the person. The processor system may be arranged to compute the quantity of a reflex receptive field of the person by determining a two-dimensional interpolation of the determined measures of nociceptive withdrawal reflex thresholds for the plurality of stimulation positions. The processor system may be arranged to compute the quantity of a reflex receptive field by applying said two-dimensional interpolation of the determined measures of nociceptive withdrawal reflex thresholds for the plurality of stimulation positions. For example, a value indicative of an area of the person's reflex receptive field may be generated. For example, a reflex receptive field quantity value determined as a fraction of the person's foot size may be generated.

According to one embodiment the stimulator comprises a plurality of individually controllable stimulator electrodes spatially distributed to provide stimulation on different positions on the person's skin, such as the person's foot sole. The individually controllable stimulator may be controllable by the processor system in accordance with a spatial selection algorithm.

According to one embodiment the electromyographic sensor comprises a plurality of surface electromyographic electrodes. The electromyographic sensor may for example comprise at least 3 surface electromyographic electrodes. The system may be arranged to apply a spatial filtering technique to output signals of a plurality of spatially different positioned surface electromyographic electrodes. The system may be arranged to apply a cross talk reduction technique to output signals of a plurality of spatially differently positioned surface electromyographic electrodes to reduce cross talk between concurrently active muscles.

According to one embodiment the reflex detection algorithm involves calculating an interval peak z-score in response to the set of electromyographic related parameter(s) representative of muscle activity.

According to one embodiment the reflex detection algorithm involves performing a conduction velocity analysis on the set of electromyographic related parameter(s) representative of muscle activity.

According to one embodiment, the stimulation intensity algorithm comprises an up-down staircase method, such as an interleaved up-down staircase method.

### BRIEF DESCRIPTION OF THE DRAWINGS

The above, as well as additional objects, features and advantages of the present inventive concept, will be better understood through the following illustrative and non-limiting detailed description of preferred embodiments of the present inventive concept, with reference to the appended drawings. In the drawings like reference numerals will be used for like elements unless stated otherwise.

FIG. 1 is a schematic illustration of a system which may be used to determine an NWR-T of a person.

FIG. 2 illustrates a general overview of a method for determining the NWR-T of a person.

FIG. 3 illustrates in more detail a method for determining the NWR-T of a person.

FIG. 4 illustrates a method for quantifying an RRF of a person.

FIG. 5 schematically illustrate examples of raw EMG traces for the tibialis anterior muscle elicited after stimulation of the ten sites on the sole of the foot.

FIG. 6 illustrates visual representations of the mean RRF area (interpolation of the mean values at the various sites) of the 21 subjects extracted from the various RRF mappings at baseline and at reassessments both within the same session and at another individual session.

FIG. 7 shows box plots of the six different quantifications of the RRF area at baseline and at reassessments both within the same session and at another individual session. Numbers on the ordinate axis represent fraction of the sole of the foot. Numbers under the individual boxes indicate the number of subjects in which the RRF area was zero.

FIGS. 8a, 8b show Bland-Altman plots for within (left) and between (right) sessions comparisons for the six different quantifications of the RRF area. Numbers on both axes represent fraction of the sole of the foot. The dotted lines represent the mean difference between the two measurements (bias) whereas the slashed lines indicate the limits of agreement.

### DETAILED DESCRIPTION

Detailed embodiments of the present inventive concept will now be described with reference to the drawings.

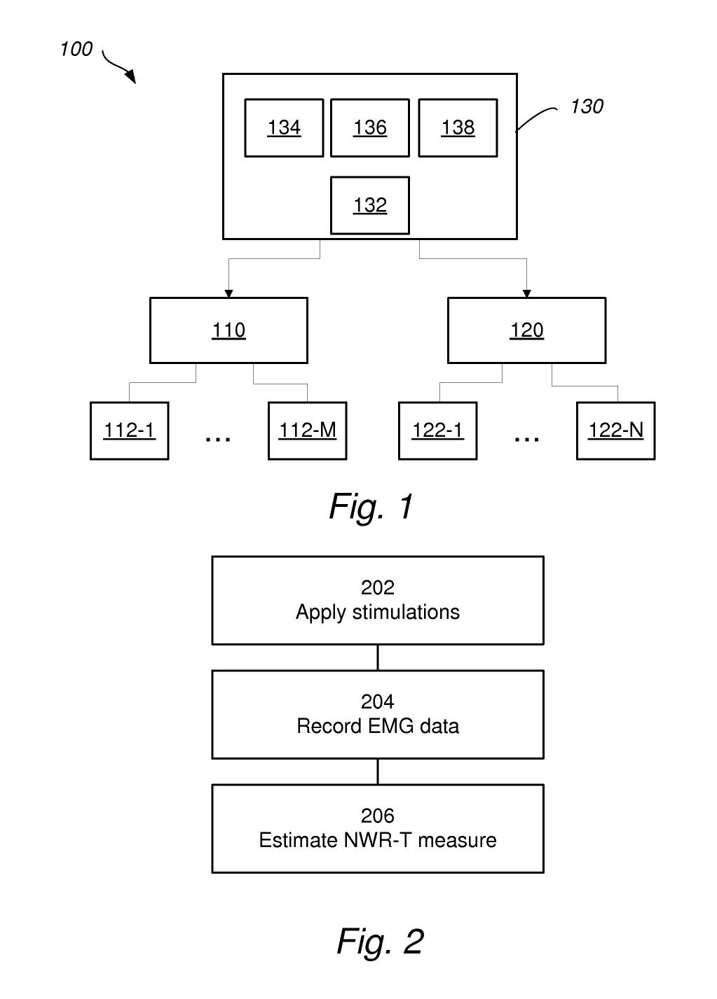

FIG. 1 illustrates a system 100 which may be used to determine an NWR-T of a test subject, which in the following may be interchangeably referred to as a person. The system 100 comprises a stimulator 110, an electromyographic sensor 120 and a processor system 130.

The stimulator 110 is arranged to generate a stimulation in response to a control signal provided by the processing system 130. In response to receiving the control signal from the processing system 130, the stimulator 110 is arranged to generate a stimulation of an intensity level indicated by the control signal. The stimulator 110 is arranged to provide the stimulation to the test subject as an electrical stimulation via a set of stimulator electrodes 112-1, . . . , 112-M which are galvanically connected to the stimulator 110 (e.g. using one or more conducting wires).

The one or more electrodes of the set of stimulator electrodes 112-1, . . . , 112-M may be skin electrodes arranged to be attached to the skin of the person, e.g. using an adhesive. Each electrode of the set of stimulator electrodes 112-1, . . . , 112-M may further include an anode-cathode configuration. The anode and the cathode may preferably be arranged in a single unit, such that only a single unit needs to be placed in contact with skin of the person. Further possible implementations and designs of skin electrodes are however per se well-known to the person skilled in the art and will therefore not be further elaborated upon in the present disclosure.

For the purpose of generating a stimulation, the stimulator 110 includes a controllable electrical pulse generator such as a controllable current pulse generator. The pulse generator is arranged to generate and output an electrical stimulation signal in accordance with the control signal. The stimulation may include a single current pulse or a train of two or more current pulses. The pulse(s) may be either square or non-square pulses, unipolar or bipolar pulses. The stimulator 110 may adjust the intensity level of the stimulation signal by generating the pulse(s) with a maximum amplitude indicated by the control signal. As will be described in greater detail below, during a test on a person the stimulator 110 will provide a number of stimulations to the person with varying intensity levels of the stimulation. As a non-limiting example, a single stimulation may include one or more current pulse trains each including a number of individual current pulses (e.g. 1-10) having an amplitude in the range of 0-50 mA and being applied at a repetition frequency of 100-500 Hz. For most given test subjects, there is a threshold current (which exact value is individual for the test subject) in the range 1-50 mA at and above which a stimulation provokes a spinal cord withdrawal reflex in the test subject.

The set of stimulator electrodes 112-1, . . . , 112-M may include any number of electrodes, such as one, two, three or more. While a greater number of electrodes may enable more extensive testing of a subject (as will be described below) it may for some applications however suffice to use only a single electrode (e.g. for determining an NWR-T based on single stimulation site). In case the set of stimulator electrodes 112-1, . . . , 112-M includes more than one electrode the stimulator 110 preferably includes switching circuitry arranged to forward the stimulation (e.g. the pulse train) to a selected electrode of the set of electrodes 112-1, . . . , 112-M. As will be described in more detail below, an electrode of the set of stimulator electrodes 112-1, . . . , 112-M may be selected using a spatial selection algorithm.

As a test subject receives a stimulation having an intensity level sufficient to provoke the spinal cord withdrawal reflex, muscle activity will ensue. The electromyographic sensor 120 (or shorter the EMG sensor 120) of the system 100 is arranged to sense an EMG signal of the test subject and output EMG data representative of muscle activity of the person in response to a stimulation provided by the stimulator 110.

The EMG sensor 120 includes a set of surface electromyographic electrodes 122-1, . . . , 122-N, or shorter EMG electrodes. The electrodes of the set of EMG electrodes 122-1, . . . , 122-N may be connected to a respective input of the EMG sensor 120 (e.g. by conducting wires). Additionally, the EMG sensor 120 may include a surface reference electrode, i.e. an electrode configured as a reference electrode, and also connected to an input of the EMG sensor 120.

For example, the set of EMG electrodes 122-1, . . . , 122-N may include a first and a second electrode wherein the EMG sensor 120 may be arranged to sense and output data representing a single-differential (SD) EMG signal. The first and the second electrode may be positioned along the muscle fibers of a muscle of interest (e.g. the ipsilateral tibialis anterior, TA, muscle) wherein activity of the muscle may be sensed. Advantageously, the set of electrodes 122-1, . . . , 122-N includes three measurement electrodes arranged in a tri-polar electrode configuration wherein the EMG sensor 120 via the three measurement electrodes and the above-mentioned surface reference electrode may be arranged to sense and output data representative of a double-differential (DD) and two (SD) EMG signals. For example, the three measurement electrodes may be positioned along a muscle of interest spaced apart by an appropriate distance (e.g. along the ipsilateral TA muscle) and the reference electrode may be positioned at a reference position (e.g. the ipsilateral knee).

The EMG sensor 120 may further include sampling circuitry for sampling and digitizing (e.g. using an analog-to-digital-converter) the EMG signals received by the set of EMG electrodes 122-1, . . . , 122-N. The EMG sensor 120 may further include circuitry for performing appropriate amplification and filtering of sensed EMG signals using techniques which per se are well-known in the art. The EMG sensor 120 may also include circuitry arranged to generate and output the EMG data representing the sensed EMG signal(s).

The processor system 130 is connected to the stimulator 110 and to the EMG sensor 120. The processor system 130 is arranged to generate and output a set of control signals to the stimulator 110, the set of control signals being adapted to control the stimulator 110 to provide a set of stimulations with different stimulation intensity levels to the test subject, wherein at least one stimulation of said set of stimulations has an intensity level arranged to provoke a spinal cord withdrawal reflex in the test subject. The set of control signals may be determined in a manner which will be described in detail below by the processing system 130 executing “a stimulation intensity algorithm” schematically indicated as the functional block 134 in FIG. 1. The processor system 130 is further arranged to receive EMG data from the EMG sensor 120.

The processor system 130 is arranged to process the stimulation levels of the set of stimulations provided to the person and the EMG data representative of muscle activity of the person in response to each stimulation to determine a measure of the NWR-T of the test subject. The NWR-T may be determined in a manner which will be described in detail below by the processing system 130 executing “a threshold calculation algorithm” schematically indicated as the functional block 136 in FIG. 1.

The processor system 130 may include a processor 132 (such as a micro processor or a central processing unit, CPU), a memory and an I/O interface (not shown in FIG. 1). The processor 132, the memory and the I/O interface may be connected to each other, e.g. via a data bus, and arranged to communicate data between each other. The I/O interface may further be arranged to provide an interface towards the stimulator 110 and the EMG sensor 120 for outputting control signals and receiving EMG data. The I/O interface may be a wired serial interface (such as USB). However the processor system 130 may also be arranged to communicate with the stimulator 110 and the EMG sensor 120 via a wireless interface (such as Bluetooth or WLAN) provided the stimulator 110 and the EMG sensor 120 include a respective wireless communication interface. The memory of the processor system 130 may be a volatile memory, e.g. a Random Access Memory (RAM) or a flash memory etc. Preferably, the memory includes a program section and a data section, wherein the program section may store software instructions and the data section may store data to be used in inter alia the algorithms described below. The processor 132 may be arranged to execute software instructions stored in the program section of the memory and implementing the algorithms 134 and 136. However, program instructions may also be stored as firmware in a non-volatile memory such as a ROM. The software instructions may be written in one or more programming or scripting languages such as C, C++, Java, Python, Perl to name a few. The software instructions may also be stored as a computer program product on a tangible non-transitory computer-readable storage medium (such as a solid-state, a magnetic or an optical storage medium accessible by the processor system 130). The software instructions may be arranged to be executed by the processor 132, e.g. when downloaded into the memory of the processing system 130. As an alternative to software-based implementations, the processor system 130 may be implemented directly in hardware for example by dedicated circuitry and/or application specific integrated circuit(s) (ASICS).

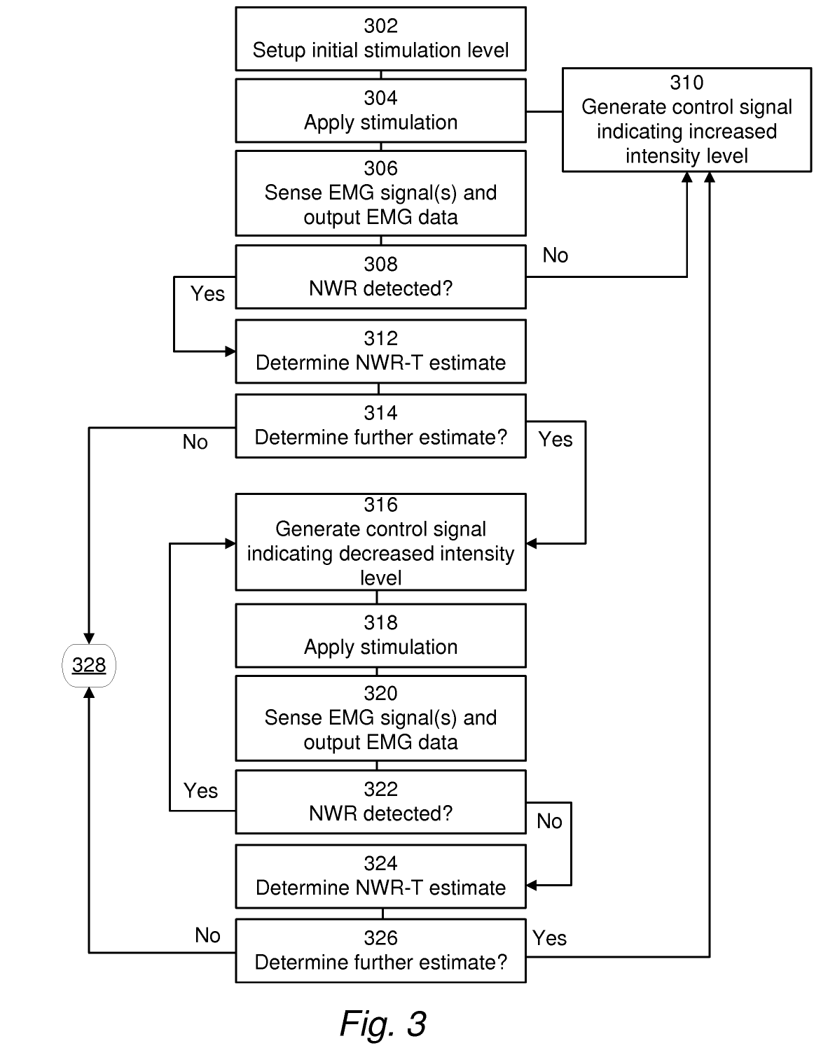

FIG. 2 illustrates a general overview of a method for determining the NWR-T of a test subject. The method comprises applying a set of a plurality of stimulations to a person with different stimulation intensity levels, step 202.

Each stimulation may be generated by the stimulator 110 and provided to the test subject. The stimulation may be provided to the test subject via the set of stimulator electrodes 112-1, . . . , 112-M. Each electrode of the set of stimulator electrodes 112-1, . . . , 112-M may be arranged in contact with a respective skin portion of the test subject. In case the set of electrodes 112-1, . . . , 112-M includes more than one electrode the stimulation may be provided to the test subject via a selected one of the set of stimulator electrodes 112-1, . . . , 112-M, e.g. selected in accordance with the spatial selection algorithm performed by the processor system 130.

The stimulation intensity level of each stimulation of the set of stimulations may be determined by the processor system 130 in accordance with the stimulation intensity algorithm 134, wherein the set of stimulations comprises at least one stimulation having an intensity level which is sufficient for provoking a spinal cord withdrawal reflex in the test subject.

The method further comprises recording EMG data representative of muscle activity of the person in response to each stimulation of the plurality of stimulations, step 204.

The EMG sensor 120 may sense an EMG signal of the test subject in response to each stimulation. The EMG signal may be sensed by the EMG sensor 120 via the set of EMG electrodes 122-1, . . . , 122-N. Each electrode of the set of EMG electrodes 122-1, . . . , 122-N may be arranged in contact with a respective skin portion of the test subject, along a muscle of interest. The EMG sensor 120 may output EMG data representative of an activity of the muscle of interest in response to said stimulation. The EMG data may be received by the processor system 130.

The method further comprises processing the stimulation intensity levels and the electromyographic data according to estimate a measure of the NWR-T of the test subject. The measure of the NWR-T may be estimated by the processor system 130 in accordance with the threshold calculation algorithm 136.

The measure of the NWR-T may be stored by the processor system 130, e.g. by the processor 132 in the data section of the associated memory for further analysis. In case the set of stimulator electrodes 112-1, . . . , 112-M includes more than one electrode the processor system 130 may preferably also store electrode selection data associating the estimated measure of the NWR-T with the electrode of the set of stimulator electrodes 112-1, . . . , 112-M via which the stimulation was applied. Additionally, the estimated measure of the NWR-T (and if applicable the electrode selection data) may be provided to a display device, such as a monitor, connected to the processor system 130, to allow for visual inspection of the result.

The processor system 130 may determine the set of control signals and the related intensity levels using, what may be referred to as, an “up-down staircase method”: The processor system 130 provides a (first) sequence of control signals for each stimulator electrode of the set of stimulator electrodes 112-1, . . . , 112-M, wherein, in each sequence, a stimulation intensity level is gradually increased until the processor system 130 receives EMG data representative of muscle activity in response to a last applied stimulation of the respective (first) sequence. The initial stimulation intensity level of each respective (first) sequence of control signals may be a predetermined intensity level (such as 1 mA) which is common for all stimulator electrodes. The stimulation intensity levels may be increased in a step-wise manner by a predetermined (first) step size (such as 2 mA). The “up staircase” branch may be followed by a “down staircase” branch. The processor system 130 provides a (second) sequence of control signals for each stimulator electrode of the set of stimulator electrodes 112-1, . . . , 112-M, wherein, in each sequence a stimulation intensity level is gradually decreased until the processor system 130 receives EMG data representative of absence of muscle activity in response to a last applied stimulation of the respective (second) sequence. The initial stimulation intensity level of each respective (second) sequence of control signals may be the intensity level of the last applied stimulation of the respective (first) sequence (at which muscle activity was detected, which stimulation intensity level may be unique for each stimulator electrode). The stimulation intensity levels may be decreased in a step-wise manner by a predetermined (second) step size (such as 1 mA).

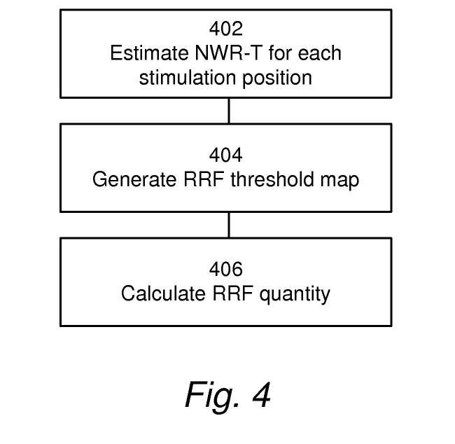

FIG. 3 illustrates in detail a method for determining the NWR-T of a person in further detail. To further facilitate understanding, the method will now be described in detail in relation to a single stimulator electrode 112-1.

The processor system 130 initiates the stimulation intensity algorithm 134 wherein an initial intensity level of a stimulation is setup, step 302. The initial intensity level may be user-supplied level (e.g. entered via the I/O interface of the processor system 130). The initial intensity level may also be a predetermined initial intensity level, stored in the processor system 130 (e.g. in the data section of the memory associated with the processor 132). By way of example the initial intensity level may be 1 mA.

The processor system 130 provides a control signal to the stimulator 110. The control signal includes information indicating the initial intensity level setup in step 302. In response to the control signal, the stimulator 110 generates a stimulation signal with the intensity level indicated by the control signal (e.g. 1 mA) and applies the stimulation signal to the test subject via the electrode 112-1, step 304.

The EMG sensor 120 senses an EMG signal of the test subject and outputs EMG data, step 306. If the EMG sensor 120 includes three or more EMG electrodes and a reference electrode the EMG sensor 120 may, as described above, sense more than one EMG signal and accordingly output EMG data representing each of the sensed EMG signals. In the event the intensity level of the stimulation is sufficient for provoking a spinal cord withdrawal reflex in the test subject muscle activity will ensue. The muscle activity will in turn influence the EMG signal(s).

The EMG data is received and analyzed by the processor system 130 employing the threshold calculation algorithm 136. Accordingly the processor system 130 determines whether the intensity level of the stimulation applied in step 304 was sufficient for provoking the spinal cord withdrawal reflex in the test subject. In other words, the processor system 130 determines whether the stimulation caused activation or elicitation of the NWR, step 308.

In the event that the processor system 130 positively determines that the intensity level of the stimulation applied in step 304 was sufficient for provoking the spinal cord withdrawal reflex in the test subject, the method proceeds to step 312. In the event that the processor system 130 determines that the intensity level of the stimulation in step 304 not was sufficient for provoking the spinal cord withdrawal reflex, the method proceeds to step 310.

In step 312, the processor system 130 determines the intensity level of the stimulation applied in step 304 as an estimate measure of the NWR-T of the person, step 312. The processor system 130 may accordingly store said intensity level as an estimated measure of the NWR-T. Hence, the threshold calculation algorithm 136 may also be referred to as a reflex detection algorithm for estimating a measure of the NWR-T in response to a reflex detection.

Different schemes for determining whether the intensity level of the stimulation applied to the test subject in step 304 was sufficient for provoking the spinal cord withdrawal reflex in the test subject are possible:

According to one example, the processor system 130 may determine a peak level of each of the one or more EMG signals, from the EMG data, and compare the peak level(s) to a (respective or common) detection threshold (which may be predetermined and configured based on experience or a priori knowledge). If each of the determined peak level(s) exceed the (respective or common) detection threshold(s), the processor system 130 may store the intensity level of the applied stimulation as an estimated measure of the NWR-T. Otherwise, the processor system 130 may conclude that the intensity level of the stimulation not was sufficient for provoking the spinal cord withdrawal reflex.

According to another example, the processor system 130 may determine a peak level of each of the one or more EMG signals and determine whether the peak level(s) deviates from a (respective) baseline mean of the EMG signal(s) (calculated for a pre-stimulation time interval of an appropriate duration) by more than a (respective or common) deviation threshold (which may be predetermined and configured based on experience or a priori knowledge). If the determined peak level(s) present a deviation exceeding the (respective or common) deviation threshold(s), the processor system 130 may store the intensity level of the stimulation as an estimated measure of the NWR-T. Otherwise, the processor system 130 may conclude that the intensity level of the stimulation not was sufficient for provoking the spinal cord withdrawal reflex.

According to another example, the processor system 130 may determine a respective interval peak z-score for each of the one or more EMG signals using the EMG data. The interval peak z-score for an EMG signal may be calculated using equation 1:

$$z\_score = \frac{reflex window peak - baseline mean}{baseline standard deviation} \qquad ( equation 1 )$$

The reflex window peak may be defined as the peak level of the EMG signal level during a post-stimulation time interval (e.g. in the range of 80-150 ms). The baseline mean and the baseline standard deviation may be calculated from EMG signal level during a pre-stimulus interval of an appropriate duration (such as 70 ms). If the interval peak z-score for each of the one or more EMG signals exceed a (respective or common) threshold, the processor system 130 may store the intensity level of the applied stimulation as an estimated measure of the NWR-T. The threshold may be a predetermined threshold, set based on experience or a priori knowledge. Available research suggests that an interval peak z-score of at least 12 indicates activation of the NWR [11, 25]. However, depending on the particulars of the test subject and the measurement setup it may in some situations be preferable to use a smaller or greater interval peak z-score threshold.

It may be understood that, to facilitate peak level detection of the EMG signal(s), the EMG signal(s) may be rectified by the EMG sensor 120 wherein the EMG data output by the EMG sensor 120 may be representative of the rectified EMG signal(s). As may be understood by the person skilled in the art, it would also be possible to omit the rectification by the EMG sensor 120 and instead apply a data transformation to the EMG data (either at the EMG sensor 120 or at the processor system 130) such that the transformed EMG data corresponds to rectified EMG signal(s).

If the set of electrodes 122-1, . . . , 122-N of the EMG sensor 120 includes three or more electrodes EMG electrodes and a reference electrode, the EMG sensor 120 may, as described above, be arranged to sense and output data representative of a DD EMG signal and two SD EMG signals. In that case, additional analysis may be performed by the processor system 130 in the event that the processor system 130 makes a positive determination regarding activation of the NWR in response to the stimulation applied in step 304: More specifically, the additional analysis may include performing a conduction velocity analysis of the EMG data. The conduction velocity analysis may include calculating cross-correlations of the SD EMG signals (i.e. in the digital domain using the EMG data). Optional high pass filtering (using a cut-off frequency of for example 80 to 100 Hz) of the SD EMG signals may be performed prior to calculating the cross-correlations. The cross-correlations may be normalized by the product of the norm of each of the two SD EMG signals. From the resulting cross-correlogram, the processor system 130 may determine the conduction velocity (CV), defined as the temporal displacement of the peak, and the maximum value of the normalized cross-correlation. If both the CV and the maximum value of the normalized cross-correlation exceed respective muscle specific thresholds (which may be predetermined based on the desired sensitivity and accuracy of the test) it is likely that increased peak levels of the EMG signals are due to cross-talk between muscles and not due to muscle activity in the monitored muscle. Accordingly, the processor system 130 may compare the CV and the maximum value of the normalized cross-correlation to a set of muscle specific thresholds. If both thresholds of the set are exceed the processor system 130 may determine that the stimulation 304 did not result in an activation of the NWR and proceed to step 310. The thresholds for the CV and the maximum value of the normalized cross-correlation may for example be in the range 30-70 m/s and 0.80-0.90, respectively. In particular, for the ipsilateral TA muscle threshold values of 34 m/s and 0.80, respectively, may be used.

In step 310, the processor system 130 (due to concluding that the intensity level of the stimulation applied in step 304 was insufficient for provoking the NWR) generates a further control signal including information indicating an increased intensity level, in relation to the initial intensity level. The intensity level may be increased by an amount defined by a first step size parameter. The first step size may be user-supplied (e.g. entered via the I/O interface of the processor system 130). The first step size may also be predetermined and stored in the processor system 130 (e.g. in the data section of the memory associated with the processor 132). By way of example the first step size may be 2 mA.

In response to the control signal, the stimulator 110 generates a further stimulation signal with the intensity level indicated by the control signal (e.g. 3 mA) and outputs the stimulation signal to the test subject via the stimulator electrode 112-1, step 304. The method thereafter proceeds as discussed in connection with step 306 and 308 above by the EMG sensor 120 sensing one or more EMG signals of the test subject and outputs EMG data. The EMG data is received and analyzed by the processor system 130 employing the threshold calculation algorithm 136. Accordingly the processor system 130 determines whether the increased intensity level of the stimulation was sufficient for provoking the spinal cord withdrawal reflex in the test subject. In the event that the processor system 130 determines that the intensity level of the stimulation in step 304 not was sufficient for provoking the spinal cord withdrawal reflex, the method again proceeds to step 310 wherein a further iteration of step 304, 306 and 308 is performed. According to the method, the loop formed by step 304, 306, 308 and 310 is iterated until a positive determination is made in connection with step 308 wherein the method proceeds to step 312. In each further iteration of the loop formed by step 304, 306, 308 and 310 the intensity level indicated by the information in the control signal provided by the processor system 130 to the stimulator 110 is increased by the first step size in relation to the former iteration. The processor system 130 may be arranged to delay the output of the control signal to the stimulator 110 to achieve a delay between consecutive stimulations. The delay may be configurable and may for example be in the range of 3-15 s.

In the event that the processor system 130 positively determines that the increased intensity level (which may have been increased one or more times by the first step size) of the last stimulation applied in step 304 was sufficient for provoking the spinal cord withdrawal reflex in the test subject, the method proceeds to step 312. In line with the above, the processor system 130 accordingly determines the intensity level of the last stimulation as an estimated measure of the NWR-T of the person. The processor system 130 may accordingly store said intensity level as an estimated measure of the NWR-T.

Optionally, an additional method step may be performed following a negative determination at step 308 prior to step 310. The additional method step may include determining whether a predetermined maximum stimulation level has been reached (for example 50 mA). If not the method may proceed to step 310. If the predetermined stimulation level has been reached the processor system 130 may determine the intensity level of the last stimulation (which hence will be equal to the predetermined maximum stimulation level) as an estimated measure of the NWR-T of the person and thereafter proceed to step 314. Thereby subjecting the person to intolerable stimulation levels may be avoided.

The portion of the method in FIG. 3 described thus far may be referred to as an “up-branch” of the staircase method. The method may further comprise a “down-branch”:

Following step 312, the method may comprise determining whether a further estimate of a measure of the NWR-T of the person should be determined, step 314. The decision may be taken by the processor system 130 evaluating a criterion. The criterion may be whether a predetermined number of measures of the NWR-T of the person have been estimated. The predetermined number may be in the range of 1-10, as a non-limiting example. In the event that the predetermined number is 1 it follows that only the up-branch will be performed. The processor system 130 may maintain a counter for this purpose. The counter may be incremented each time an NWR-T is estimated.

In response to determining that the predetermined number of measures of the NWR-T not have been estimated, the processor system 130 in step 316 generates a further control signal including information indicating a decreased intensity level, in relation to the intensity level of the last stimulation applied in step 304. The intensity level may be decreased by an amount defined by a second step size parameter. The second step size may be equal to the first step size. However, the second step size may also be smaller or greater than the first step size. Similar to the first step size, the second step size may be user-supplied (e.g. entered via the I/O interface of the processor system 130) or a predetermined step size, stored in the processor system 130 (e.g. in the data section of the memory associated with the processor 132). By way of example the second step size may be 1 mA.

In response to the control signal, the stimulator 110 generates a further stimulation signal with the (reduced) intensity level indicated by the control signal and applies the stimulation signal to the test subject via the stimulator electrode 112-1, step 318. The method thereafter proceeds to step 320 and 322. In step 320 one or more EMG signals are sensed and EMG data is output by the EMG sensor 120, in complete analogy with step 306 discussed above. In step 322 the EMG data is received and analyzed by the processor system 130 employing the threshold calculation algorithm 136. Accordingly the processor system 130 determines whether the reduced intensity level of the stimulation was sufficient for provoking the spinal cord withdrawal reflex in the test subject. In the event that the processor system 130 determines that the intensity level of the stimulation in step 318 was sufficient for provoking the spinal cord withdrawal reflex, the method returns to step 316 wherein a further iteration of steps 318, 320 and 322 is performed. The loop formed by steps 316, 318, 320 and 322 is iterated until a negative determination is made in connection with step 322 wherein the method proceeds to step 324. In each further iteration of the loop formed by steps 316, 318, 320 and 322 the intensity level indicated by the information in the control signal provided by the processor system 130 to the stimulator 110 is decreased by the second step size.

In the event that the processor system 130 determines that the reduced intensity level (which may have been decreased one or more times by the second step size) of the last stimulation applied in step 318 was not sufficient for provoking the spinal cord withdrawal reflex in the test subject, the method proceeds to step 324. In step 324, the processor system 130 determines the intensity level of the stimulation applied prior to the last stimulation applied in step 318 as a further estimated measure of the NWR-T of the person. The processor system 130 may accordingly store said intensity level as a further estimated measure of the NWR-T.

Following step 324, the method may comprise determining whether even further estimated measures of the NWR-T of the person should be determined, step 326. Step 326 is completely analogous to step 314. Accordingly, the decision may be taken by the processor system 130 evaluating a criterion such as whether the predetermined number of measures of the NWR-T of the person have been estimated.

In response to determining that the predetermined number of measures of the NWR-T not have been estimated, the up-branch of the method may be repeated wherein one or more further iterations of steps 310, 304, 306 and 308 are performed. Again, the loop formed by step 304, 306, 308 and 310 is iterated until a positive determination is made in connection with step 308 wherein the method proceeds to step 312. In each further iteration of the loop formed by step 304, 306, 308 and 310 the intensity level of the stimulation provided to the test subject is increased by a step size in relation to the former iteration. The step size may be equal to the first step size. However, preferably a smaller step size may be used during the second time the up-branch of the method is performed. For example, the second time (and beyond) the up-branch is performed the step size may be 0.5 mA.

In step 312, the processor system 130 analogous to the previous description determines the increased intensity level of the last stimulation applied in step 304 as a further estimated measure of the NWR-T of the person. The processor system 130 may store said increased intensity level as a further estimated measure of the NWR-T.

If, in step 314, it is determined that even further estimated measures of the NWR-T of the person should be determined, the down-branch of the method may be repeated wherein one or more further iterations of steps 316, 318, 320 and 322 are performed. The decision may be taken by the processor system 130 evaluating a criterion. Again, the loop formed by step 316, 318, 320 and 322 is iterated until a negative determination is made in connection with step 322 wherein the method proceeds to step 324. In each further iteration of the loop formed by steps 316, 318, 320 and 322 the intensity level of the stimulation provided to the test subject is decreased by a step size in relation to the former iteration. The step size may be equal to the second step size. However, preferably a smaller step size may be used during the second time the down-branch of the method is performed. For example, the second time (and beyond) the down-branch is performed the step size may be 0.5 mA.

Following a negative outcome of the determination in either step 314 or in step 326 the method proceeds to step 328 wherein a final estimate of the measure of the NWR-T may be calculated. At this point, a set of one or more estimated measures of the NWR-T of the person have been determined and stored by the processor system 130. The processor system 130 may in step 328 for example calculate the final estimate as the mean value of all or a subset of the set of one or more estimated measures of the NWR-T. The final estimate may also be calculated as the median of all or a subset of the set of one or more estimated measures of the NWR-T. The subset may for example be selected to exclude one or more extreme values of the estimates. The final estimate of the NWR-T may be stored by the processor system 130, e.g. by the processor 132 in the data section of the associated memory for further analysis. The final estimate may be stored in a data set associated with the test subject. The processor system 130 may further output the final estimate for display on a display device connected to the processor system 130.

Using a stimulator 110 comprising more than one stimulator electrode, such as two, three or more, enables determining measures of NWR-T for more than one stimulation position on the person, such as a plurality of positions on the foot sole of the test subject. The processor system 130 may execute a spatial selection algorithm wherein the stimulations positions (i.e. the stimulator electrodes of the set of stimulator electrodes 112-1, . . . , 112-M) during testing of the person may be selected in a randomized order. The processor system 130 may control via which stimulator electrode each stimulation is to be applied by indicating the selected stimulator electrode in the control signal provided to the stimulator 110. The stimulator 110 may in turn control the switching circuitry in accordance with the indication included in the control signal. The final estimate of the NWR-T for each stimulation position may be stored by the processor system 130, e.g. by the processor 132 in the data section of the associated memory for further analysis. The final estimates may stored in a data set associated with the test subject.

With reference to FIG. 3, the up-down staircase method may be applied in turn for each stimulation position (i.e. stimulator electrode) wherein measures of NWR-T may be individually estimated for each stimulation position. The order in which the stimulation positions are stimulator may be randomly selected using the spatial selection algorithm. Alternatively, the up-down staircase method may be applied in an interleaved fashion for the stimulation positions wherein the processor system controls the stimulator 110 to apply a stimulation of a respective intensity level via each one of the stimulator electrodes 112-1, . . . , 112-M, in a random order, and thereafter to apply a stimulation of an increased or decreased respective intensity level (i.e. during “the up branch” increased in relation to the previous intensity level applied via the respective stimulator electrode, and during “the down branch” decreased in relation to the previous respective intensity level applied via the respective stimulator electrode) via each one of the stimulator electrodes 112-1, . . . , 112-M, in a random order. Hence the stimulation level at each stimulation position is selected in accordance with an independently running up/down branch. Thus, at any given instance of a test some stimulation positions may be on a respective up branch and others on a respective down branch.

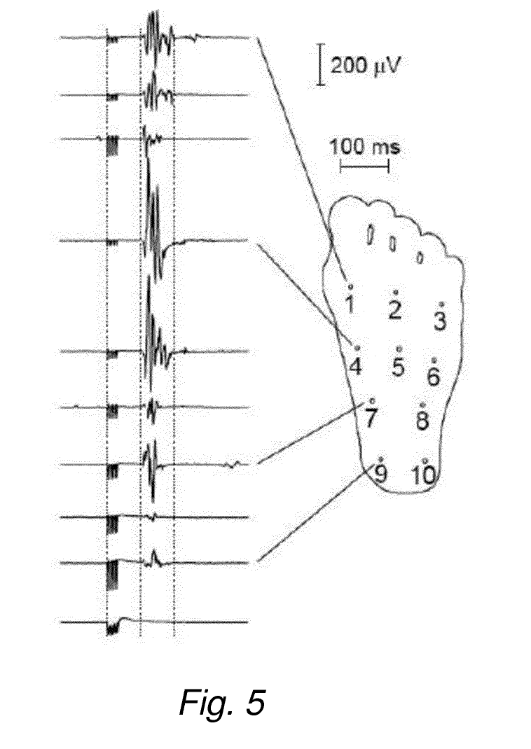

FIG. 4 illustrates a method for determining a quantity of an RRF of a test subject using a plurality of measures of NWR-T of the test subject. The steps of the method may for example be implemented by a RRF determination algorithm, schematically indicated as the functional block 138 in FIG. 1. According to the method, a measure of an NWR-T is estimated for each stimulation position of a plurality of stimulation positions of the test subject, step 402. The NWR-T measures may be estimated in accordance with the above methods by the processor system 130 and stored in a memory for further processing as described above.

Based on the estimated NWR-T measures the processor system 130 generates an RRF threshold map, step 404. The map may relate each estimated NWR-T measure to a respective stimulation position. Each respective stimulation position may be associated with a set of coordinates in a coordinate system. Each stimulation position may for example be associated with a pair of coordinates (x, y) in a Cartesian coordinate system. The set of coordinates may be user-defined based on the actual positioning of the stimulator electrodes 112-1, . . . , 112-M. The coordinates may for example be entered via an user input device (such as a mouse, a keyboard a touch screen or the like) connected to an I/O interface of the processor system 130. The processor system 130 may optionally calculate a two-dimensional interpolation from the estimated NWR-T measures. Thereby an interpolated continuous RRF threshold map may be obtained which relates an NWR-T measure to each point within the area defined by the collection of the stimulation positions. Additionally, the processor system 130 may generate an inverse RRF threshold map (discrete or interpolated). The one or more maps may be stored by the processor system 130, e.g. in a data set associated with the test subject. The processor system 130 may generate a graphical representation of the map for display on a display device. The graphical representation of the map may for example be displayed on a display connected to the processor system 130.

Based on any of the above-described maps, the processor system 130 may calculate a quantity representative of the RRF, step 406. For example, from the non-inversed threshold map an RRF quantity may be calculated as the fraction of the tested portion of the test subject presenting estimated an NWR-T which is lower than a threshold. The threshold may be an absolute predetermined or user-defined threshold or a relative threshold defined by the minimum NWR-T in the map plus two times the standard deviation of all the interpolated threshold values. The quantity may be stored by the processor system 130, e.g. in a data set associated with the test subject. Provided the surface area of the skin portion including the stimulation positions is known and stored by the processor system 130, a quantity representing the actual surface area having an NWR-T (or inverse NWR-T) exceeding a threshold of may be calculated (e.g. by multiplying the surface area with the fraction meeting the criterion). Other examples of RRF quantities which the processor system 130 may calculate will be given below.

On the basis of either a measure of the NWR-T or an RRF quantity (estimated in accordance with the above) a person may be classified with respect to pain hypersensitivity. The processor system 130 may, following estimation of a measure of the NWR-T or an RRF quantity compare the result thereof to a reference value. In the event that the threshold value is exceeded the person may be classified as suffering from pain hypersensitivity and otherwise, if the threshold value not is exceeded, the person may be classified as not suffering from pain hypersensitivity. The processor system 130 may for example set a flag (e.g. in the data memory associated with the processor 132) to true if the threshold value is exceeded and to false if the threshold value not is exceeded. The flag may be associated with a data set including the results of the testing of the person. The processor system 130 may further output the result of the classification for display on a display device connected to the processor system 130.

To further facilitate understanding, an example study employing the system and methods in accordance with the present inventive concept will now be described:

### Subjects

Twenty one healthy volunteers (age: 18-35 years) participated in the study. Written informed consent was obtained from all subjects prior to participation and the Declaration of Helsinki was respected. The study was approved by the local ethical committee (approval number VN—20130006).

### Experimental Procedure

The experiment consisted of two similar sessions; a single session and a double session performed in randomized order with a 48±1 h intersession interval. In the single-session, mapping of NWR sensitivity and NWR probability were carried out for the tibialis anterior (TA) muscle in accordance with the procedures previously published by Neziri et al. [23] and Manresa et al. [21], respectively (5 repetitions of painful stimuli were applied). In addition, individual NWR-T were identified for 10 stimulation sites under the sole of the foot using the above described procedures. In the double session, the same procedures for mapping of NWR sensitivity, NWR probability and identification of NWR-T were carried out twice (1 h pause between estimates), with the single exemption that pain thresholds for the individual stimulation sites were identified only ones, i.e. the same set of painful stimulation intensities were applied in the two consecutive mappings of both NWR sensitivity and NWR probability. The two previously published methods for RRF mapping constitutes two different analyses of the same data (i.e. only one sequence of painful stimulations was applied per assessment). The order of painful stimulation and identification of NWR-T was randomized between subjects but kept constant between both intra- and inter-session reassessments for each individual subject.

Initial Setup

The subjects had been required to refrain from caffeine, nicotine, alcohol and strenuous exercise for at least 4 h and from analgesic medication for 24 h prior to the experiment. Thick epidermal layers on the sole of the foot were ground off using a callus remover in order to reduce the effect of variation in skin thickness before mounting stimulation electrodes. During the experiment, the subject was placed in supine position with back support inclined 135 degrees relative to the horizontal level. A pillow was placed under the knees, resulting in a knee flexion of 45 degrees. Prior to actual data acquisition, the subject was thoroughly familiarized with electrical stimulation to reduce any effects of arousal or anxiety.

### Stimuli for NWR Elicitation—Electrical Stimulation

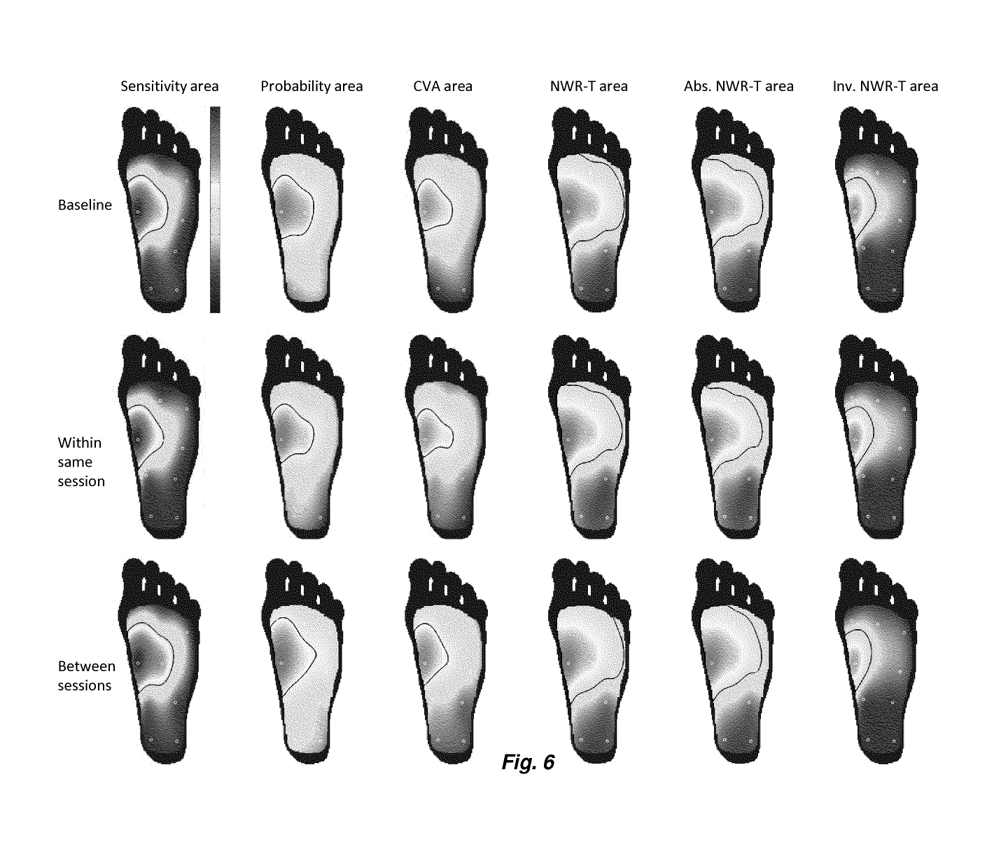

Ten surface stimulation electrodes (20×15 mm, type 700, Ambu A/S, Denmark) were mounted in a non-uniform grid on the plantar side of the foot to elicit the NWR (FIG. 5). One large common anode (70×100 mm, Pals, Axelgaard, USA) was placed on the dorsum of the foot to ensure that the stimulus was perceived as coming from the sole of the foot. Any of the 10 stimulation electrodes was moved slightly in case the evoked sensation indicated direct nerve trunk stimulation (i.e. radiating sensation). Each stimulus consisted of a constant current pulse train of five individual 1 ms pulses delivered at 200 Hz (felt as a single stimulus) by a computer controlled electrical stimulator (Noxitest IES 230, Aalborg, Denmark).

### NWR Recording—EMG

Activity in the ipsilateral tibialis anterior (TA) muscle was recorded using surface EMG. Three surface electrodes (type 720, Ambu A/S, Denmark) were placed in parallel on the shaved skin along the overall orientation of the muscle with an interelectrode distance of 2 cm. A common reference electrode (70×100 mm, Pals, Axelgaard, USA) was placed on the ipsilateral knee. The tri-polar electrode configuration fed three separate amplifiers for simultaneous recording of one double differential (DD) and two single-differential (SD) EMG signals. The signals were amplified, filtered (10-500 Hz), sampled (10 kHz) and stored (1 s window including 200 ms pre-stimulation).

### Automated NWR Detection

Automated NWR detection was performed online using custom made software based on evaluation of interval peak z-score [25] and a recently published method denoted Conduction Velocity Analysis (CVA). CVA was specifically designed to distinguish genuine reflexes from EMG crosstalk and enable reflex detection with an improved specificity [15]. A NWR was detected if the interval peak z-scores from all three recorded EMG signals indicated the occurrence of a NWR and this EMG response could not be attributed to crosstalk as evaluated by the CVA method.

Interval Peak z-Score

The EMG signals were rectified and their interval peak z-score were calculated in accordance with equation 1 presented above. An interval peak z-score exceeding 12 indicates a NWR [11,25]. The reflex window was defined as the 80-150 ms post-stimulus interval whereas the baseline mean and standard deviation was calculated from the 70 ms pre-stimulus interval.

Conduction Velocity Analysis (CVA)

The two SD EMG signals were high pass filtered (10th order Butterworth with zero phase delay) with cut-frequency at 80 hertz, whereupon cross-correlation of the two recordings were performed. The cross-correlations were normalized by the product of the norm of each of the two signals. From the resulting cross-correlogram, two features were extracted; the conduction velocity (CV) defined as the temporal displacement of the peak and the maximal value of the normalized cross-correlation. CVA was performed on all sweeps were the interval peak z-score of all three EMG signals exceeded 12 (indicating a reflex) but the activity were attributed to just crosstalk (and no reflex was detected) if both the CV and the maximal value of the normalized cross-correlation were above a set of fixed thresholds of 34 m/s and 0.80 respectively, (see [15] for detailed description of the method).

### Staircase Method for Identification of NWR Thresholds

The NWR-T was determined for each of the ten stimulation sites using an interleaved up-down staircase method: for each site, the stimulation intensity was initially set to 1 mA and then increased in steps of 2 mA until the NWR was detected, whereupon the intensity was reduced in steps of 1 mA until the NWR was no longer detected. The intensity was subsequently increased and decreased in steps of 0.5 mA until a total of 3 ascending and 3 descending estimations of the NWR-T had been achieved. The final NWR-T was then considered the mean value of the last 2 ascending and 2 descending estimations. The staircases for the individual stimulation sites were interleaved, meaning that all 10 sites were stimulated in random order (double blinded) before any site was stimulated again whereby the thresholds for the 10 sites were identified simultaneously. Stimulations were applied with a 3-5 s inter-stimulus interval via a computer controlled electrical relay. If the stimulation intensity reached 50 mA or the stimulation intensity at a site became intolerable for the subject, stimulation of this specific site was discontinued and the NWR-T at that site was defined as the last stimulation intensity applied.

### Data Analysis

RRF Mapping

The NWR-T identified for each of the 10 stimulation sites were used to produce two novel and completely objective topographical mappings of the RRF sensitivity. The approximate location of the ten stimulation sites were marked on a graphical representation of a standardized foot and a two-dimensional interpolation of the identified NWR-T values were performed to constitute a RRF threshold map. To avoid basing any findings on extrapolated values, the mapping was restricted to the interpolated part of the image (the part of the image encompassed by the electrodes). A similar but reciprocal mapping that better illustrates the topographical distribution of NWR sensitivity was produced by carrying out another two-dimensional interpolation performed on the inverse values of the identified NWR-T (1 divided by the individual thresholds). Features describing the area (fraction of the entire interpolated mapping) of the RRF were extracted from both types of mapping in order to allow quantitative statistical analysis of the RRF mappings. A total of three novel RRF area features were extracted from the two different mappings: One from the inverse threshold map and two from the non-inversed threshold map applying relative and absolute thresholds, respectively. A RRF area feature (NWR-T area) based on a relative threshold, inspired by Neziri et al. (2009) [23], were calculated from the non-inversed threshold map as the fraction of the sole of the foot encompassing NWR-T lower than a threshold defined by the minimum NWR-T in the map plus two times the standard deviation of all the interpolated threshold values. The second feature (Abs. NWR-T area) extracted from the non-inversed threshold map using an absolute threshold were calculated as the fraction of the sole of the foot with NWR-T lower than a fixed threshold of 25 mA (50% of the maximum possible threshold). From the other mapping—the inverse threshold map, a single RRF area feature (Inv. NWR-T area) was calculated as the fraction of the sole of the foot with values exceeding a threshold defined by the peak value in the map minus two standard deviations.

Maps of NWR sensitivity (NWR magnitude) and NWR probability were produced for the individual subjects, and two features (Sensitivity area′ and ‘Probability area’) quantifying the RRF area were extracted as described by Neziri et al. [23] and Manresa et al. [21], respectively. An additional type of mapping was produced by repeating the procedure used for probability maps but including CVA in the preprocessing associated with the NWR detection. From this mapping another novel RRF area feature (CVA area) was derived by applying the same threshold as for the probability maps.

### Reliability

Bland-Altman agreement analysis was used to assess both the within-session and between-session reliability of the various RRF area features. Bland-Altman agreement analysis compares the average and the difference between data measured in two different sessions, from which the limits of agreement (LOA) can be derived as the average difference±1.96 times the standard deviation of the differences. The LOA delimits the range within which 95% of the differences between two repeated measurements may be expected to lie. Closely related hereto, the coefficient of repeatability (CR) is defined as the value below which 95% of the absolute differences between measurements in two single sessions may be expected to lie [7]. The difference between two individually sampled and calculated means constitute the estimated bias, which means that the upper and lower LOA can be expressed as bias±CR. LOA is a measure of absolute reliability and is hence not affected by the range of measurements in use and it is reported in the same units as the underlying data. The results of the Bland-Altman agreement analysis is expressed as CR and bias. Paired t-tests (with a significance level of 0.05) were applied to determine if the calculated biases were significantly different from zero in order to evaluate if there exist any significant difference in the means from the paired measures.

Another way to compare the reliability of different methods is to compare the sample sizes needed to detect a clinically relevant effect on the RRF area. Thus, sample sizes for parallel (Nₚ) and crossover (N_{c}) designs were calculated for each RRF area feature. The desired significance level (α) was set to 0.05 by convention and the desired power (1-β) was set to 0.8 whereas the clinically relevant effect (E) was set to 0.10 (RRF area expressed as a fraction of the sole of the foot) [5,24]. For a parallel study design the sample size (Nₚ) was estimated as:

$${N}_{p} = \frac{15.6 {σ}^{2}}{{E}^{2}}$$

where σ is the standard deviation of the RRF areas [16]. For a crossover study design the sample size (N_{c}) was estimated as:

$${N}_{c} = \frac{15.6 {σ}^{2} ( 1 - ρ )}{{E}^{2}}$$

where ρ is Pearsons correlation coefficient estimated between two measures on the same subject [16].

### Results

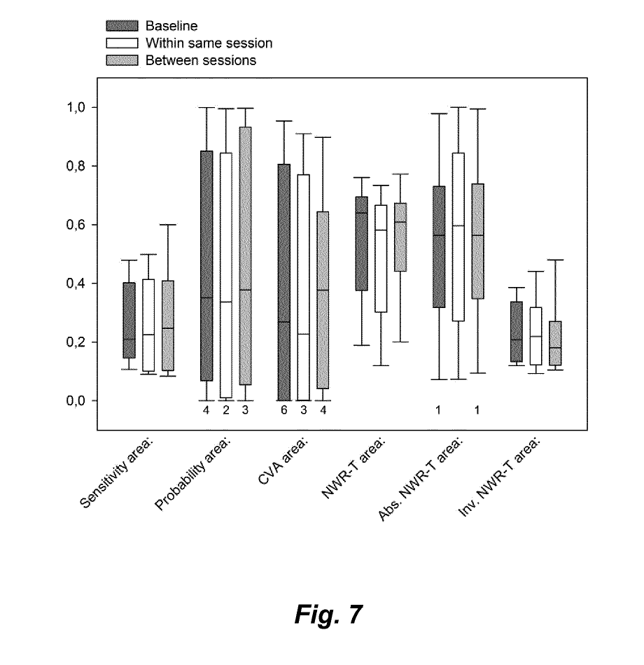

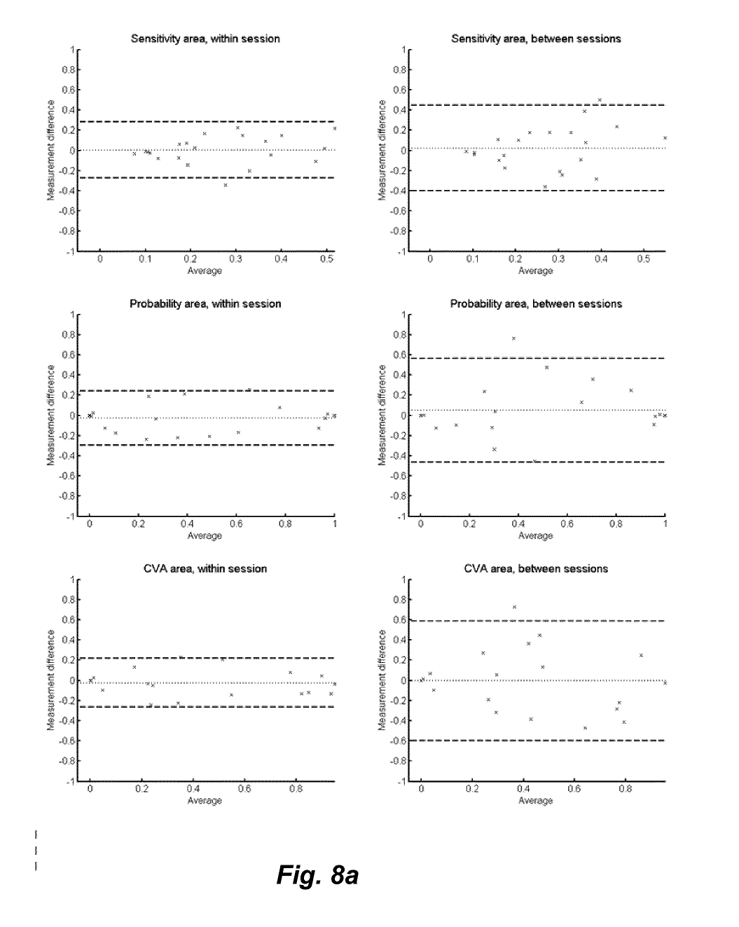

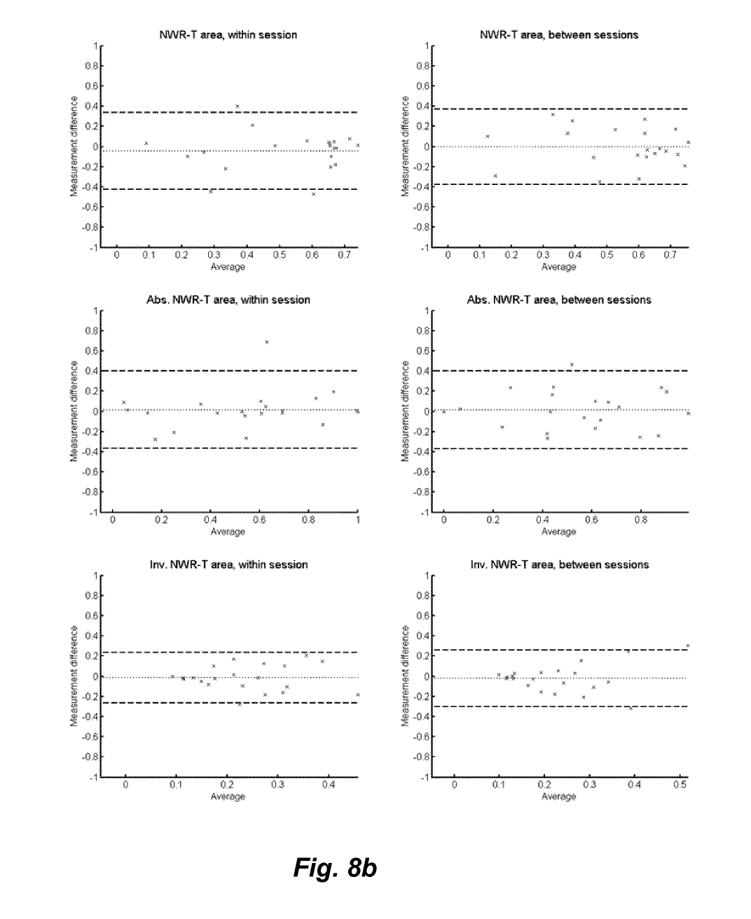

Visual representations of the mean RRF area of the 21 subjects can be seen in FIG. 6, whereas FIG. 7 shows boxplots of the various features quantifying the RRF at the three different time instances. As depicted in FIG. 7, the three RRF features involving an absolute threshold (Probability area, RRF CVA area and Abs. NWR-T area) all resulted in at least one case of an extracted area of zero. The calculated CRs, listed in table 1, indicates that the RRF features extracted from mappings produced from data acquired using previously published methods (Sensitivity area, Probability area and CVA area) all showed a better within-session reliability than between-session reliability (performed on different days). Conversely, the three novel features based on the NWR-T each exhibited a similar measure of reliability within and between sessions. Of these, the Inv. NWR-T area demonstrated superior reliability both within and between sessions. None of the calculated biases were significantly different from zero. Bland-Altman plots are shown in FIGS. 7 and 8.

TABLE 1
Coefficient of repeatability (CR) and bias for the various RRF area
features

| | |
|---|---|
| Within-session | Between-session |
| reliability: | reliability: |

| Feature: | CR: | Bias: | CR: | Bias: |
|---|---|---|---|---|
| Sensitivity area: | 0.28 | 0.01 | 0.43 | 0.02 |
| Probability area: | 0.27 | −0.03 | 0.52 | 0.05 |
| CVA area: | 0.24 | −0.02 | 0.59 | −0.00 |
| NWR-T area: | 0.38 | −0.04 | 0.38 | −0.01 |
| Abs. NWR-T area: | 0.38 | 0.02 | 0.39 | 0.02 |
| Inv. NWR-T area: | 0.25 | −0.02 | 0.28 | −0.02 |

Hypothetical sample sizes, listed in table 2, also indicate that the Inv. NWR-T area constituted the most reliable RRF quantification of the six features. Sample sizes calculated for a crossover study design followed the pattern of the CR; each of the three NWR-T area features demonstrated a similar level of reliability (similar sample sizes) within and between sessions, whereas the remaining features all demonstrated poorer between-session reliability than within-session reliability. All of the three NWR-T area features required a smaller sample size than any of the existing RRF measures to detect a clinically relevant effect in a crossover study design involving more than one session. For sample sizes calculated for a parallel study design, very large sample sizes were associated with area features where one or more RRF areas of zero were detected (cf. FIG. 7).

TABLE 2
Hypothetical sample sizes needed to detect a clinically relevant effect
(of 0.1) in a parallel (Nₚ) and crossover (N_{c}) study design, respectively.

| | |
|---|---|
| Within-session | Between-session |
| reliability: | reliability: |

| Feature: | Nₚ | N_{c} | Nₚ | N_{c} |
|---|---|---|---|---|
| Sensitivity area: | 36 | 15 | 42 | 36 |
| Probability area: | 232 | 15 | 243 | 53 |
| CVA area: | 203 | 12 | 185 | 69 |
| NWR-T area: | 72 | 29 | 65 | 28 |
| Abs. NWR-T area: | 144 | 29 | 124 | 30 |
| Inv. NWR-T area: | 21 | 13 | 27 | 16 |

### DISCUSSION

In the above, a new objective method for mapping and quantification of reflex receptive fields of the nociceptive withdrawal reflex in humans has been presented. The method does not involve any subjective assessment by neither the subject nor the investigator and provides excellent within- and between-session reliability.

A total of six different features quantifying the RRF area were extracted from five different topographical mappings produced using two different data acquisition approaches. Three RRF features (Sensitivity area, Probability area and CVA area) were extracted from three corresponding mappings produced using a previously published approach where properties (size or probability) of NWRs elicited by a set of fixed stimulation intensities applied throughout the sole of the foot were interpolated. The three remaining RRF features (NWR-T area, Abs. NWR-T area and Inv. NWR-T area) were extracted from two novel mappings produced by interpolation of NWR-T detected at various stimulation sites across the sole of the foot.

Both the presented Sensitivity areas and Probability areas seems slightly smaller than previously reported in healthy subjects [5,21,24] but not more than what may be explained by the relative large variation within previous reports. The remaining RRF features have never previously been reported.

### Assessment of Reflex Receptive Fields—What are we Measuring?

Despite being an intrinsic defense mechanism, the NWR does not constitute a fixed stimulus-response system—equal stimulations may cause very different levels of reflex responses. The NWR may instantly be modulated by supraspinal centers through descending inhibition and facilitation during cognitive tasks like distraction or attention [6,18]. Descending control also plays a pivotal role in maintaining a normal excitability level and functional organization of spinal NWR pathways [3,13]. Increased spinal excitability has been documented in patients with various clinical conditions by evaluation of NWR-T [2,9,19,24,28]. Such a state of hyperexcitability may reflect central sensitization characterized by an enhanced responsiveness of nociceptive neurons in the central nervous system to normal and/or sub-threshold afferent input causing hyperalgesia and allodynia [20]. Several studies have found alterations and expansion of the receptive fields of dorsal horn neurons due to central changes caused by persistent barrage in large unmyelinated peripheral fibres [8,14]. The sensitivity of the RRF is probably encoded by putative reflex interneurons located in the deep part of the dorsal horn [27]. Central sensitization causing expansion of the receptive fields of dorsal horn neurons may consequently also lead to expansion of RRFs, rendering assessment of RRFs suitable to assess central sensitization. Enlarged RRFs in a group of chronic pain patient compared to healthy controls has already been shown [5]. This indicates that a state of hyperexcitability caused by persistent pain indeed entail expansion of receptive fields of not only dorsal horn neurons but also of the NWR.

### Objectivity

The NWR is strongly dependent on stimulus intensity, and under appropriate conditions (e.g. adequate stimulation parameters and posture), NWRs involving a particular muscle in the lower limb in humans can be evoked from the entire sole of the foot [1]. Hence, in order to enable meaningful RRF quantification, stimulation intensities for elicitation of the NWR at the various sites across the sole of the foot need to be comparable across subjects but individually normalized. In previous methods, this is achieved by titrating the stimulation intensities to individual pain thresholds in an attempt to establish an equal afferent input to the nociceptive system when the various sites are stimulated [1]. However, that requires the subject to make reliable subjective assessments of the unnatural electrical stimuli. Many subjects find this task very difficult and give vague or hesitant answers when directly asked if a specific stimulus is perceived as being painful, in some subjects these indistinct responses even continues after high intensity stimuli with obvious physical indications of pain (e.g. facial or oral expressions). The task of identifying subjective pain thresholds at the various sites is furthermore complicated by the fact that the quality of the perceived sensation due to the electrical stimulation differs across the sole of the foot [10]. In order to circumvent this subjective assessment, the novel methods for RRF quantification introduced in this paper, assess the spinal nociceptive excitability at the various sites by objective identification of NWR-T as an alternative to assessment of NWR response size to specific stimulation intensities. Both the NWR and to a greater extend subjective pain ratings may be modulated by several factors. Some desired properties included in the assessment of spinal hyperexcitability (e.g. supraspinal control increasing facilitation and/or decreasing inhibition and hereby modulating the effects of sensitization) and others considered noise in most experimental protocols (e.g. attention/distraction or emotional state including anxiety). However, by eliminating any subjective involvement in the assessment, a substantial source of variability may be eliminated rendering a more reliable method.

### Reliability

The NWR-T has been shown to be a reliable measure suitable for both experimental and clinical use [4,22,29]. However, reports on the reliability of RRF quantifications are extremely limited and only the within-session reliability has yet been investigated. Biurrun Manresa et al. [4] found that both RRF Sensitivity area and RRF Probability area measurements showed a high level of within-session reliability in low back pain patients. The present results support these findings and suggest that a sample size of 15 subjects is adequate to detect a RRF area change of 0.1 in a crossover study design if both pre- and post-intervention assessments are performed within one session. However, if the two assessments are carried out on different days, an increase in variability demands much larger sample sizes of 36 and 53 for Sensitivity areas and Probability areas, respectively. A corresponding difference in within- and between-session reliability for these previous published RRF quantification methods can be seen in the reported coefficients of repeatability. These inconsistences in reliability are practically non-existing for the new objective RRF quantification features based on NWR-T, suggesting that these constitute more reliable measures in general with improved between-session reliability in particular. However, the present results indicate that the Inv. NWR-T area entails superior reliability both within and between sessions implying that this new feature may outright be the most reliable method available for quantification of RRFs.

### Applicability of the Various Methods

The present results indicate that the new objective method based on detection of NWR-T can reliably be applied at any one point in time. The existing methods evaluating the NWR size or probability to a set of fixed stimulation intensities do demonstrate a similar level of reliability, but only if the same set of fixed stimulation intensities are applied in two individual assessments. This means that, in terms of reliability, the existing methods mapping NWR size and probability are well-suited to investigate a change in nociceptive excitability occurring between two assessments where the same set of fixed stimulation intensities can be applied (e.g. in a pre/post assessment design). However, if the experimental design does not allow for this (e.g. in a cross-sectional research design) the reliability of the existing methods are markedly reduced (in the same range as those presented as the between-session reliability). In contrast, the novel NWR-T methods do not depend on any fixed set of stimulation intensities and thereby constitute more reliable alternatives. In general, the superior reliability both within the same session and between sessions performed on different days of the Inv. NWR-T area feature allows it to be used in more complex research designs.

A practical limitation common for the RRF area features involving an absolute threshold (Probability area, CVA area and Abs. NWR-T area) is that the identified probability or threshold may not reach the fixed threshold and a RRF area of zero is calculated even if some NWRs are detected. Besides the obvious consequence hereof of not being sensitive to sub-threshold changes, this has a negative effect on the reliability of the methods. These negative consequences are negligible considering the reliability in a crossover study design but cause a radical increase in the sample size necessary to detect a clinically relevant effect size in a parallel study design.

Because the new objective method for RRF quantification does not rely on any subjective assessments performed by the subject, it may be applied in a wider range of populations than the previously published methods. With no requirements to the subject's ability to perform a valid assessment of perception, the new method may for example be applied on subjects with cognitive deficits or subjects suffering from sensory loss e.g. due to spinal cord injuries.

### CONCLUSIONS

In the above a novel objective method for acquisition and quantification of reflex receptive fields that enables reliable assessment of nociceptive excitability has been described. Existing alternatives involves subjective elements and shows a reduced reliability between sessions carried out on individual days. Conversely, the newly introduced method demonstrates good reliability both within and between sessions. This experiment provides evidence for an improved reliability but future studies investigating the sensitivity of the method to confirm its overall superiority would be of value. However, the present invention contributes to the potential to use RRF as an objective biomarker for pain hypersensitivity in clinical and experimental pain research.

In the above the inventive concept has mainly been described with reference to a limited number of examples. However, as is readily appreciated by a person skilled in the art, other examples than the ones disclosed above are equally possible within the scope of the inventive concept, as defined by the appended claims. For example, although in the above, the determination of the set of control signals has been described as being performed by “a stimulation intensity algorithm”, and the estimation of the NWR-T as being performed by “a threshold detection algorithm” or “a reflex detection algorithm”, this should not be construed as the operations attributed to these “algorithms” necessarily being implemented by individual or separate functional units, blocks or modules. In fact, the different algorithms may be understood as merely a logical distribution and grouping of the operations which may be used to implement the various aspects and embodiments in accordance with the present inventive concept. Moreover, although illustrated as separate components or units in FIG. 1, it should be noted that it would be equally possible to implement the stimulator 110, the EMG sensor 120 and the processing system 130 in common circuitry and/or in a common enclosure.

### LIST OF REFERENCES

In the above disclosure, one or more numbers between a pair of brackets “[ ]” refer to a correspondingly numbered prior art document in the following list:
[1] Andersen O K. Studies of the organization of the human nociceptive withdrawal reflex. Focus on sensory convergence and stimulation site dependency. Acta Physiol (Oxf) 2007; 189 Suppl 654:1-35.
[2] Banic B, Petersen-Felix S, Andersen O K, Radanov B P, Villiger P M, Arendt-Nielsen L, Curatolo M. Evidence for spinal cord hypersensitivity in chronic pain after whiplash injury and in fibromyalgia. Pain 2004; 107:7-15.
[3] Biurrun Manresa J A, Finnerup N S, Johannesen I L, Biering-Sorensen F, Jensen T S, Arendt-Nielsen L, Andersen O K. Central sensitization in spinal cord injured humans assessed by reflex receptive fields. Clin Neurophysiol 2014; 125:352-362.
[4] Biurrun Manresa J A, Neziri A Y, Curatolo M, Arendt-Nielsen L, Andersen O K. Test-retest reliability of the nociceptive withdrawal reflex and electrical pain thresholds after single and repeated stimulation in patients with chronic low back pain. Eur J Appl Physiol 2011; 111:83-92.
[5] Biurrun Manresa J A, Neziri A Y, Curatolo M, Arendt-Nielsen L, Andersen O K. Reflex receptive fields are enlarged in patients with musculoskeletal low back and neck pain. Pain 2013; 154:1318-1324.
[6] Bjerre L, Andersen A T, Hagelskjaer M T, Ge N, Morch C D, Andersen O K. Dynamic tuning of human withdrawal reflex receptive fields during cognitive attention and distraction tasks. Eur J Pain 2011; 15:816-821.
[7] Bland J M, Altman D G. Measuring agreement in method comparison studies. Stat Methods Med Res 1999; 8:135-160.
[8] Cook A J, Woolf C J, Wall P D, McMahon S B. Dynamic receptive field plasticity in rat spinal cord dorsal horn following C-primary afferent input. Nature 1987; 325:151-153.
[9] Courtney C A, Lewek M D, Witte P O, Chmell S J, Hornby T G. Heightened flexor withdrawal responses in subjects with knee osteoarthritis. J Pain 2009; 10:1242-1249.
[10] Frahm K S, Morch C D, Grill W M, Andersen O K. Experimental and model-based analysis of differences in perception of cutaneous electrical stimulation across the sole of the foot. Med Biol Eng Comput 2013; 51:999-1009.
[11] France C R, Rhudy J L, McGlone S. Using normalized EMG to define the nociceptive flexion reflex (NFR) threshold: further evaluation of standardized NFR scoring criteria. Pain 2009; 145:211-218.
[12] French D J, France C R, France J L, Arnott L F. The influence of acute anxiety on assessment of nociceptive flexion reflex thresholds in healthy young adults. Pain 2005; 114:358-363.
[13] Harris J, Clarke R W. Organisation of sensitisation of hind limb withdrawal reflexes from acute noxious stimuli in the rabbit. J Physiol 2003; 546:251-265.
[14] Hylden J L, Nahin R L, Traub R J, Dubner R. Expansion of receptive fields of spinal lamina I projection neurons in rats with unilateral adjuvant-induced inflammation: the contribution of dorsal horn mechanisms. Pain 1989; 37:229-243.
[15] Jensen M B, Manresa J A, Frahm K S, Andersen O K. Analysis of muscle fiber conduction velocity enables reliable detection of surface EMG crosstalk during detection of nociceptive withdrawal reflexes. BMC Neurosci 2013; 14:39-2202-14-39.
[16] Julious S A. Sample sizes for clinical trials with normal data. Stat Med 2004; 23:1921-1986.
[17] Latremoliere A, Woolf C J. Central sensitization: a generator of pain hypersensitivity by central neural plasticity. J Pain 2009; 10:895-926.
[18] Liebermann D G, Defrin R. Characteristics of the nociceptive withdrawal response elicited under aware and unaware conditions. J Electromyogr Kinesiol 2009; 19:e114-22.
[19] Lim E C, Sterling M, Stone A, Vicenzino B. Central hyperexcitability as measured with nociceptive flexor reflex threshold in chronic musculoskeletal pain: a systematic review. Pain 2011; 152:1811-1820.
[20] Loeser J D, Treede R D. The Kyoto protocol of IASP Basic Pain Terminology. Pain 2008; 137:473-477.
[21] Manresa J A, Jensen M B, Andersen O K. Introducing the reflex probability maps in the quantification of nociceptive withdrawal reflex receptive fields in humans. J Electromyogr Kinesiol 2011; 21:67-76.
[22] Micalos P S, Drinkwater E J, Cannon J, Arendt-Nielsen L, Marino F E. Reliability of the nociceptive flexor reflex (RIII) threshold and association with Pain threshold. Eur J Appl Physiol 2009; 105:55-62.
[23] Neziri A Y, Curatolo M, Bergadano A, Petersen-Felix S, Dickenson A, Arendt-Nielsen L, Andersen O K. New method for quantification and statistical analysis of nociceptive reflex receptive fields in humans. J Neurosci Methods 2009; 178:24-30.
[24] Neziri A Y, Haesler S, Petersen-Felix S, Muller M, Arendt-Nielsen L, Manresa J B, Andersen O K, Curatolo M. Generalized expansion of nociceptive reflex receptive fields in chronic pain patients. Pain 2010; 151:798-805.
[25] Rhudy J L, France C R. Defining the nociceptive flexion reflex (NFR) threshold in human participants: a comparison of different scoring criteria. Pain 2007; 128:244-253.
[26] Rhudy J L, Martin S L, Terry E L, France C R, Bartley E J, DelVentura J L, Kerr K L. Pain catastrophizing is related to temporal summation of pain but not temporal summation of the nociceptive flexion reflex. Pain 2011; 152:794-801.
[27] Schouenborg J, Weng H R, Kalliomaki J, Holmberg H. A survey of spinal dorsal horn neurones encoding the spatial organization of withdrawal reflexes in the rat. Exp Brain Res 1995; 106:19-27.
[28] Sterling M, Hodkinson E, Pettiford C, Souvlis T, Curatolo M. Psychologic factors are related to some sensory pain thresholds but not nociceptive flexion reflex threshold in chronic whiplash. Clin J Pain 2008; 24:124-130.
[29] von Dincklage F, Hackbarth M, Schneider M, Baars J H, Rehberg B. Introduction of a continual Rill reflex threshold tracking algorithm. Brain Res 2009; 1260:24-29.

1. A system for determining a nociceptive withdrawal reflex threshold of a person, the system comprising:
a stimulator arranged to generate a stimulation in response to a control signal, wherein an intensity level of the stimulation is adjustable in accordance with information in the control signal,
an electromyographic sensor arranged to sense an electromyographic signal and output electromyographic data representative of muscle activity of the person, and
a processor system arranged for connection to the stimulator and the electromyographic sensor,
wherein the processor system is arranged to provide a set of control signals to the stimulator, the set of control signals being adapted to control the stimulator to provide a set of stimulations with different stimulation intensity levels to the person, wherein at least one stimulation of said set of stimulations has an intensity level arranged to provoke a spinal cord withdrawal reflex in the person,
wherein the processor system is arranged to receive electromyographic data representative of muscle activity of the person in response to each stimulation of said set of stimulations, and
wherein the processor system is arranged to process the stimulation intensity levels and said electromyographic data representative of muscle activity of the person in response to each stimulation to determine a measure of the nociceptive withdrawal reflex threshold of the person.

2. System according to claim 1, wherein the processor system is arranged to determine measures of nociceptive withdrawal reflex thresholds for a plurality of different stimulation positions on the person, such as a plurality of positions on the person's foot sole.

3. System according to claim 2, wherein the processor system is programmed to select the plurality of different stimulation positions in a randomized order.

4. System according to claim 2, wherein the processor system is arranged to generate an output indicative of a reflex receptive field of the person using the measures of nociceptive reflex thresholds for the plurality of different stimulation positions on the person

5. System according to any of claim 4, wherein the processor system is arranged to generate the output indicative of the reflex receptive field using a two-dimensional interpolation of the determined measures of nociceptive withdrawal reflex thresholds for the plurality of stimulation positions.

6. System according to claim 5, wherein the processor system is arranged to compute: a quantity of the reflex receptive field indicative of a surface area of a skin portion of the person including the stimulation positions which surface area presents a nociceptive reflex threshold exceeding a threshold, and/or a quantity of the reflex receptive field indicative of a fraction of a surface area of the person including the stimulation positions, which fraction presents a nociceptive reflex thresholds exceeding a threshold.

7. System according to claim 1, wherein the stimulator comprises a plurality of individually controllable stimulator electrodes arranged to be spatially distributed to provide stimulation on different positions on the person's skin, such as the person's foot sole.

8. System according to claim 7, wherein the individually controllable stimulator electrodes are controllable by the processor system in accordance with a spatial selection algorithm.

9. System according to claim 1, wherein the electromyographic sensor comprises a plurality of surface electromyographic electrodes.

10. System according to claim 9, wherein the electromyographic sensor comprises includes at least four surface electrodes, wherein three of said surface electrodes are configured as measurement electrodes and one of said surface electrodes is configured as a reference electrode.

11. System according to claim 1, wherein the processor system is arranged to, for at least a subset of said set of stimulations, calculate a respective interval peak z-score using the electromyographic data representative of muscle activity of the person in response to the respective stimulation.

12. System according to claim 1, wherein the processor system is arranged to, for at least a subset of said set of stimulations, perform a respective conduction velocity analysis of the electromyographic data representative of muscle activity of the person in response to the respective stimulation.

13. System according to claim 1, wherein the processor system is arranged to determine said set of control signals using an up-down staircase method, such as an interleaved up-down staircase method.

14. A method for determining a nociceptive withdrawal reflex threshold of a person, the method comprising:
applying a plurality of stimulations to a person with different stimulation intensity levels in accordance with a stimulation intensity algorithm, wherein the plurality of stimulations comprises at least one stimulation having an intensity level provoking a spinal cord withdrawal reflex in the person,
recording electromyographic data representative of muscle activity of the person in response to each stimulation of the plurality of stimulations, and
processing the stimulation intensity levels and the electromyographic data according to a threshold calculation algorithm to calculate a measure of the nociceptive withdrawal reflex threshold of the person.

15. A method for quantifying a reflex receptive field of a person, the method comprising calculating a plurality of nociceptive withdrawal reflex thresholds according to the method of claim 14 for a plurality of different stimulation positions of the reflex receptive field of the person, and calculating a quantity measure of the reflex receptive field of a person according to a predetermined algorithm.

16. A method for classifying a person with respect to pain hypersensitivity, the method comprising applying the method of claim 14 on the person, and comparing a result thereof with a reference value.

17. A computer program product comprising instructions arranged to perform the method according to claim 14 when executed on a processor system.
